(19) United States
(12) Patent Application Publication
(10) Pub. No.: US 2011/0263954 A1
(43) Pub. Date: Oct. 27, 2011

(54) NONINVASIVE BLOOD GLUCOSE METER

(76) Inventor: TZU-YI LIN, Taipei City (TW)

(21) Appl. No.: 12/764,141

(22) Filed: Apr. 21, 2010

(51) Int. Cl. A61B 5/1455

(52) U.S. Cl. 600/316

(57) ABSTRACT

A noninvasive blood glucose meter for testing a blood glucose level of a living organism includes a casing, a printed circuit board and a display device. The casing includes a containing space, and the printed circuit board is installed in the containing space and includes a control unit and a memory unit, and the printed circuit board is electrically coupled to a light emitting device and a light receiving device, and the light emitting device is capable of emitting a light beam, and the light receiving device is provided for receiving a reflected light beam. After the light beam emitted from the light emitting device is entered into the testing living organism, reflected from the testing living organism, and received by the light receiving device, the light beam is passed through and processed by the control unit, and then displayed on a display device and stored in the memory unit.

### BACKGROUND OF THE INVENTION

1. Field of the Invention

The present invention generally relates to blood glucose testing, and more particularly to a noninvasive blood glucose meter.

2. Description of Prior Art

In general, diabetes patients usually take a self-test of their blood glucose level to learn about the blood glucose level from the test in order to control their diet and blood glucose level, and thus a record of data taken on a regular basis can be provided as a reference for medical professionals to give medical advices and appropriate treatments.

In early stage, the blood glucose was tested by clinistix paper, whose color is compared to know about the blood glucose level. Now, a blood sample connected from a patient's finger is generally used for the test instead, and a blood glucose meter is used to read the blood sample on a test strip, such that the measured glucose blood level can be obtained from the computation performed by the blood glucose meter.

Diabetes patients usually monitor their blood glucose many times in a day, so that the way of sticking a needle into the patient's finger to collect the blood sample for the test gives more pains to the patient and increases the opportunity of getting infected.

In view of the aforementioned shortcomings of the prior art, the inventor of the present invention based on years of experience in the related industry to conduct extensive researches and experiments, and finally developed a noninvasive blood glucose meter in accordance with the present invention to overcome the shortcomings of the prior art.

### SUMMARY OF THE INVENTION

Therefore, it is a primary objective of the present invention to provide a noninvasive blood glucose meter that measures blood glucose level of living organisms by an infrared noninvasive method to avoid the pain and infection that may occur when taking a blood sample for the test.

To achieve the foregoing objective, the present invention provides a noninvasive blood glucose meter for testing a blood glucose level of a living organism, and the noninvasive blood glucose meter comprises a casing, a printed circuit board and a display device. The casing includes a containing space, and the printed circuit board is installed in the containing space and includes a control unit and a memory unit, and the printed circuit board is electrically connected to a light emitting device and a light receiving device, and the light emitting device is capable of emitting a light beam, and the light receiving device is provided for receiving a reflected light beam. After the light beam emitted from the light emitting device is entered into the testing living organism, reflected from the testing living organism, and received by the light receiving device, the light beam is passed through and processed by the control unit, and then displayed on a display device and stored in the memory unit.

Another objective of the present invention is to provide a noninvasive blood glucose meter, whose small volume allows users to carry the blood glucose meter or wear the blood glucose meter at their wrist for performing a test anytime.

Compared with the prior art, the present invention uses the light emitting device to emit an infrared light into a living organism, and the light beam reflected from the living organism is received by the light receiving device, and then computed by the control unit, so that users can know about their blood glucose level after comparison and computation take place. Since the invention no longer needs to stick a needle into a patient's finger or any part of the patient's body, the invention can avoid unnecessary pain and a possibility of getting infected. In addition, the noninvasive blood glucose meter is small and portable, so that users can wear the blood glucose meter at their wrist and take a test anytime. Obviously, the invention enhances the convenience and practicability of the blood glucose meter.

### BRIEF DESCRIPTION OF DRAWINGS

FIG. 1 is an exploded view of a noninvasive blood glucose meter in accordance with a first preferred embodiment of the present invention;

FIG. 2 is an exploded view of a noninvasive blood glucose meter in accordance with a first preferred embodiment of the present invention, viewed from another angle;

FIG. 3 is a perspective view of a noninvasive blood glucose meter in accordance with a first preferred embodiment of the present invention;

FIG. 4 is a schematic view of using a noninvasive blood glucose meter in accordance with a first preferred embodiment of the present invention; and

FIG. 5 is a schematic view of a noninvasive blood glucose meter in accordance with a second preferred embodiment of the present invention.

### DETAILED DESCRIPTION OF THE INVENTION

The technical characteristics, features and advantages of the present invention will become apparent in the following detailed description of the preferred embodiments with reference to the accompanying drawings. The drawings are provided for reference and illustration only, but not intended for limiting the present invention.

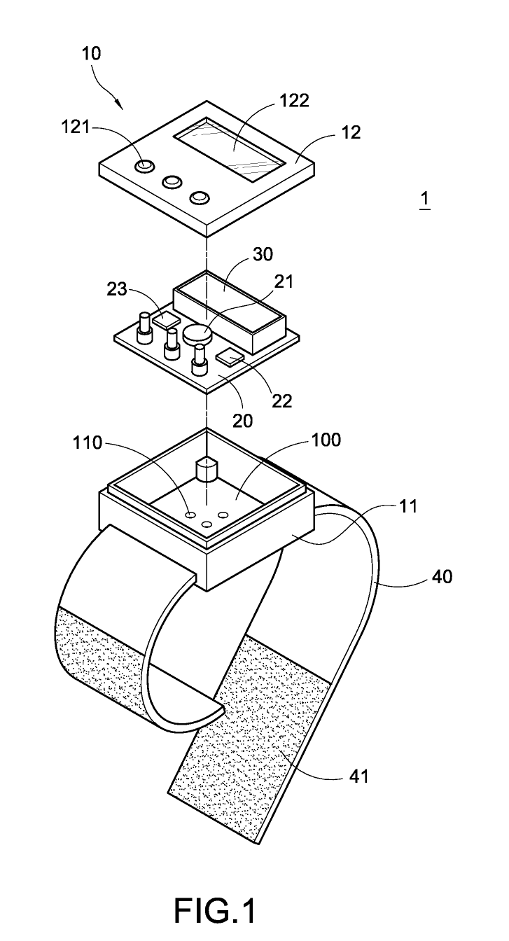

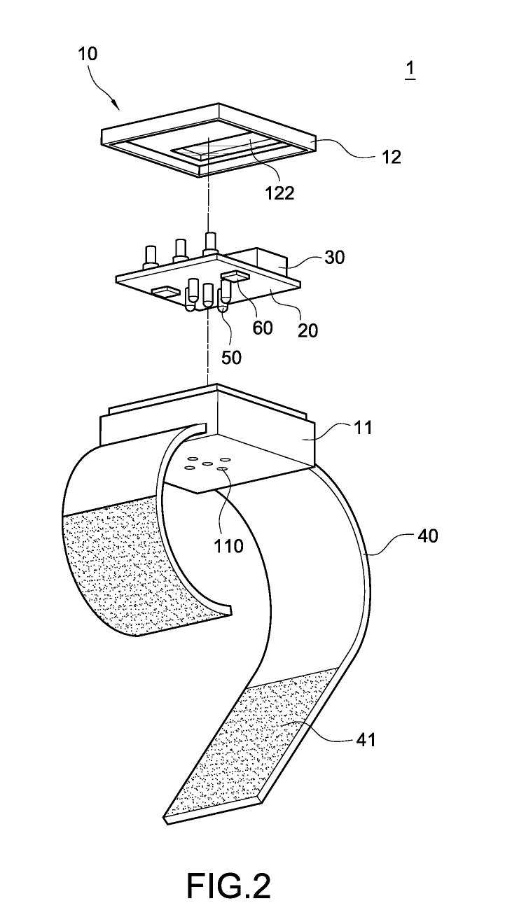

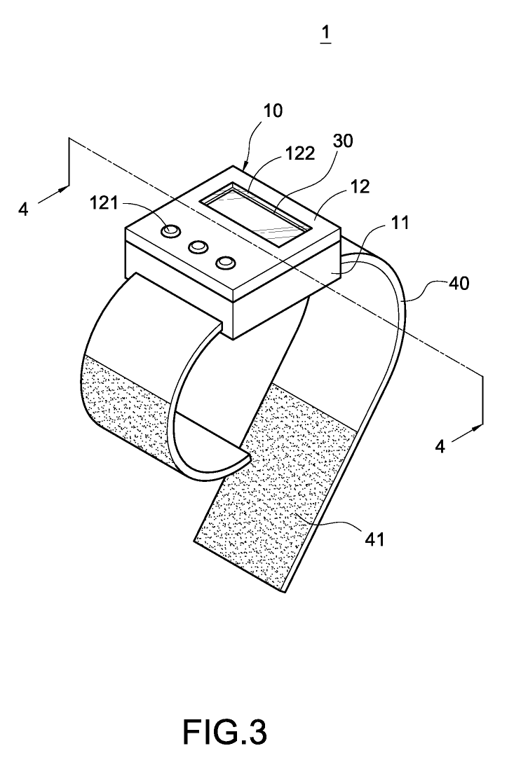

With reference to FIGS. 1 to 3 for a noninvasive blood glucose meter 1 used for testing a blood glucose level of a testing living organism in accordance with the present invention, the noninvasive blood glucose meter 1 comprises a casing 10, a printed circuit board 20 and a display device 30.

The casing 10 is formed by engaging a chassis 11 and an upper lid 12, and the chassis 11 includes a containing space 100 for containing the printed circuit board 20, and the upper lid 12 includes a plurality of press keys 121 and a transparent plate 122, and the bottom of the chassis 11 has a through groove 111 and at least one through hole 110, and a wrist band 40 is passed through the through groove 111, and the wrist band 40 includes a Velcro strip 41 provided for a user to wear the blood glucose meter 1 to a part of the user's body.

In addition, at least one light emitting device 50 and at least one light receiving device 60 are installed on the printed circuit board 20, and the light emitting device 50 is electrically coupled to the printed circuit board 20, disposed on the printed circuit board 20, and passed through the through hole 110 of the chassis 11 for emitting a light beam, wherein the light beam is an infrared light or a laser light, and the light emitting device 50 is a semiconductor laser diode for emitting the laser light, and the light receiving device 60 is electrically coupled to the printed circuit board 20 for receiving the reflected light beam emitted by the light emitting device 50.

The printed circuit board 20 includes a power supply device 21, a control unit 22 and a memory unit 23 installed thereon, and the power supply device 21 is a power source for supplying electric power required for the operation of the printed circuit board 20, and the control unit 22 has the functions of spectral analysis, processing and computation, and the memory unit 23 is provided for storing processed information of the control unit 22 and a correction curve, and the correction curve is a relation curve of a spectrum of a reflected and analyzed light beam contributed by a blood glucose factor. In addition, the printed circuit board 20 is electrically coupled to a display device 30, wherein the display device 30 of this preferred embodiment is installed on the printed circuit board 20, and the transparent plate 122 of the upper lid 12 is disposed at a position corresponding to the display device 30 provided for a user's view.

After the reflected light beam is received by the light receiving device 60, the light receiving device 60 transmits a signal to the control unit 22. After the signal is computed by the control unit 22 and compared with the correction curve stored in the memory unit 23, a blood glucose level is calculated, and the numeric value of the blood glucose level is displayed on the display device 30 and stored in the memory unit 23 for future reference and use.

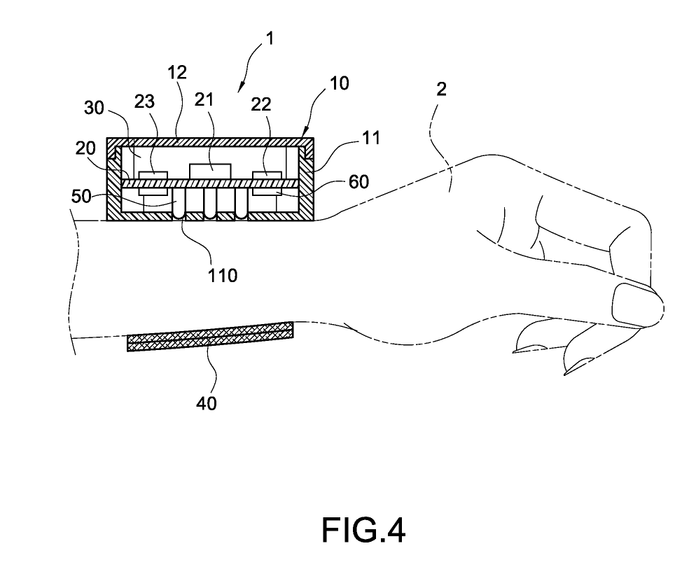

With reference to FIG. 4 for a schematic view of using a noninvasive blood glucose meter in accordance with the present invention, the wrist band 40 is wound around a patient's wrist 2 to allow the blood glucose meter 1 to perform blood glucose testing, and the light beam emitted from the light emitting device 50 is entered into the wrist 2, and a portion of the light beam is reflected from the wrist 2 and received by the light receiving device 60. After the computation and comparison performed by the control unit 22 take place, the value of blood glucose contained in the wrist 2 is calculated and displayed on the display device 30.

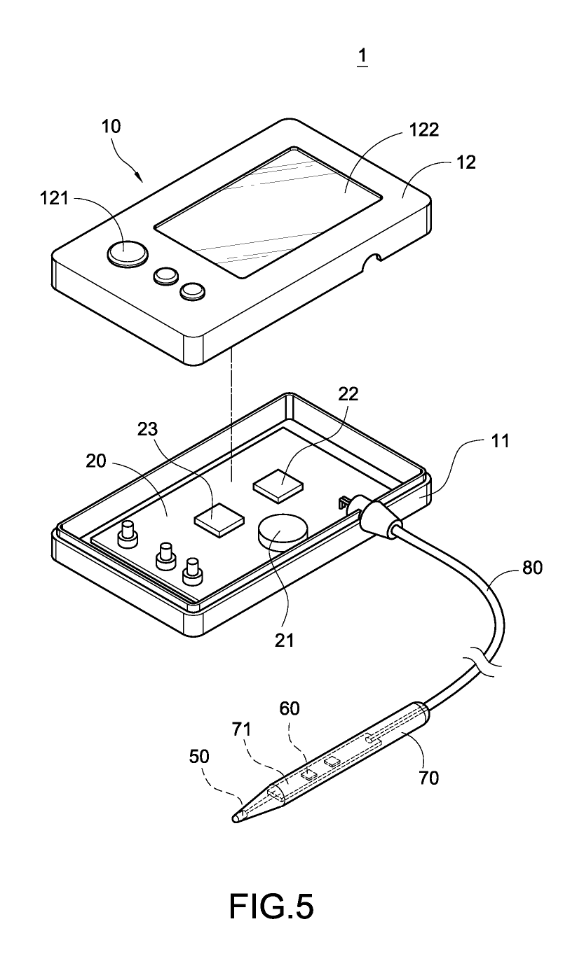

With reference to FIG. 5 for a noninvasive blood glucose meter in accordance with a second preferred embodiment of the present invention, the second preferred embodiment is substantially the same as the first preferred embodiment with the following exceptions. In the second preferred embodiment, a detection pen 70 is installed to a side of the casing 10, and the detection pen 70 includes a control circuit board 71 installed therein, and the control circuit board 71 is electrically coupled to the printed circuit board 20 by a conductive wire 80, and a front end of the control circuit board 71 is electrically coupled to the light emitting device 50, and the light receiving device 60 is installed on the control circuit board 71.

The present invention is illustrated with reference to the preferred embodiment and not intended to limit the patent scope of the present invention. Various substitutions and modifications have suggested in the foregoing description, and other will occur to those of ordinary skill in the art. Therefore, all such substitutions and modifications are intended to be embraced within the scope of the invention as defined in the appended claims.

What is claimed is:

1. A noninvasive blood glucose meter, used for testing a blood glucose level of a testing living organism, and the noninvasive blood glucose meter comprising:
a casing, having a containing space;
a printed circuit board, installed in the containing space, and including a control unit and a memory unit;
a light emitting device, electrically coupled to the printed circuit board, for emitting a light beam;
a light receiving device, electrically coupled to the printed circuit board, for receiving the light beam emitted by the light emitting device and reflected to the light receiving device; and
a display device, electrically coupled to the printed circuit board;
wherein the light beam emitted by the light emitting device and entered into the testing living organism is reflected by the testing living organism and received by the light receiving device, and information processed by the control unit is displayed on the display device and stored in the memory unit.

2. The noninvasive blood glucose meter of claim 1, wherein the casing includes a through groove provided for passing a wrist band.

3. The noninvasive blood glucose meter of claim 2, wherein the wrist band includes a Velcro strip.

4. The noninvasive blood glucose meter of claim 1, wherein the casing is formed by engaging an upper lid and a chassis.

5. The noninvasive blood glucose meter of claim 4, wherein the chassis includes a through hole, and the light emitting device is passed out from the through hole for emitting the light beam.

6. The noninvasive blood glucose meter of claim 4, wherein the upper lid includes a plurality of press buttons and a transparent plate.

7. The noninvasive blood glucose meter of claim 6, wherein the display device is installed on the printed circuit board and disposed at a position corresponding to the transparent plate.

8. The noninvasive blood glucose meter of claim 1, wherein the light emitting device and the light receiving device are installed on the printed circuit board.

9. The noninvasive blood glucose meter of claim 1, wherein the casing includes a detection pen installed on a side of the casing, and the detection pen includes a control circuit board electrically coupled to the printed circuit board.

10. The noninvasive blood glucose meter of claim 9, wherein the light receiving device is installed on the control circuit board.

11. The noninvasive blood glucose meter of claim 9, wherein the detection pen includes the light emitting device installed at a front end of the detection pen and electrically coupled to the control circuit board.

12. The noninvasive blood glucose meter of claim 1, wherein the printed circuit board further includes a power supply device for supplying electric power.

13. The noninvasive blood glucose meter of claim 1, wherein the control unit has a spectral analysis function.

14. The noninvasive blood glucose meter of claim 1, wherein the light beam is an infrared light.

15. The noninvasive blood glucose meter of claim 1, wherein the light beam is a laser light.

16. The noninvasive blood glucose meter of claim 15, wherein the light emitting device is a semiconductor laser diode.
